(19) United States
(12) Patent Application Publication
(10) Pub. No.: US 2008/0260844 A1
(43) Pub. Date: Oct. 23, 2008

(54) Solid, Oral, Microparticulate Dosage Form Which Has Been Designed To Prevent Misuse

(75) Inventors: Gerard Soula, Meyzieu (FR); Florence Guimberteau, Montussan (FR)

Correspondence Address: PATTON BOGGS LLP, 8484 WESTPARK DRIVE, SUITE 900, MCLEAN, VA, 22102, US

(73) Assignee: Flamel Technologies, Venissieux (FR)

(21) Appl. No.: 11/791,471

(22) PCT Filed: Nov. 21, 2005
(86) PCT No.: PCT/FR2005/050973

§ 371 Date: Jan. 16, 2008

(30) Foreign Application Priority Data
Nov. 24, 2004 (FR) 0452744

(51) Int. Cl. A61K 9/14; A61K 31/522

(52) U.S. Cl. 424/494; 424/489; 424/499; 424/501; 424/490; 424/497; 514/263.38

(57) ABSTRACT

The invention relates to the field of solid, oral, microparticulate dosage forms having a composition that prevents the misuse of the active pharmaceutical ingredient contained therein. The aim of the invention is to prevent the improper use of solid oral medicaments for any use other than the therapeutic use(s) officially approved by the appropriate public health authorities. More specifically, the invention relates to a solid, oral drug form which is characterised in that at least one part of the active pharmaceutical ingredient is contained in the microparticles thereof and in that the inventive form comprises anti-crushing means which are intended to impede or completely prevent the crushing of the microparticles of the active pharmaceutical ingredient, such as to preclude the misuse thereof.

### FIELD OF THE INVENTION

The present invention relates to the field of solid, oral, microparticulate pharmaceutical forms having a composition that prevents the misuse of the active pharmaceutical ingredient (AI) contained therein.

The active ingredients (AIs) under consideration are pharmaceutical AIs, for example those classified in the category of narcotic products. The latter are those for which abuse gives rise to drug addiction-related behavior.

For the purpose of the present disclosure, the expression “AI” denotes both a single active ingredient and a mixture of several active ingredients.

For the purpose of the present invention, the expression “microparticulate pharmaceutical form” is intended to mean any form in which the AI is contained in microparticles of less than 1000 microns in size. These particles containing the AI may be microcapsules for modified release of AI. In the latter case, the microcapsules are, for example, coated with a polymer film which controls the rate of release of the AI after oral administration.

The aim of the present invention is to prevent the improper use of solid oral medicaments for any use other than the therapeutic use(s) officially approved by the appropriate public health authorities. In other words, it is a question of preventing intentional or unintentional misuse of solid oral medicaments.

### SITUATION OF THE PROBLEM

Misuse is mainly encountered in the following cases:
a. addictive behavior (drug addiction, doping),
b. criminal behavior (chemical dependency),
c. use of a medicament in a manner that does not comply with the medical recommendations (posology), inadvertently or due to disabilities affecting the patient,
d. self-medication.

In case a. (or even in case b.), individuals who have the intention of misusing the solid oral medicament will generally apply themselves to making it either into a pulverulent form which can be inhaled, or into a liquid form which can be injected using a syringe.

The obtaining of an injectable liquid form from a solid oral medicament involves a step consisting of aqueous or alcoholic extraction of the targeted AI. This extraction can be preceded by crushing.

Methods of administration by inhalation or by injection are particularly suitable for drug addicts because they are methods which make it possible to accentuate the effects of the AI and which promote its absorption in the body over short periods of time. When this powder is aspirated via the nose or dissolved in water and injected, the desired doping or euphoria-inducing effects of the AI manifest themselves very rapidly and in an exacerbated manner.

The misuse of solid oral medicaments can also be observed when the medicament is chewed before being swallowed, instead of being swallowed rapidly in accordance with the posology.

The risks associated with addictive behavior (a.) and criminal behavior (b.) and with self-medication (d.) are obvious. It will be recalled that the misuse of medicaments by injection is a serious situation: the excipients may be responsible for local tissue necroses, for infections, and for respiratory and cardiac problems.

As regards abuses (c.) of the use of a medicament that are related to inattention and/or to disabilities of the patient, they can also have serious consequences. For example, the chewing, before swallowing, of forms for modified release of AI converts the medicament into an immediate-release form. Thus, at best, the medicament is ineffective after a very short period of time, and, at worst, it becomes toxic.

There clearly exists therefore a serious public health problem related to the misuse of medicaments, and in particular of solid oral medicaments.

This increasing phenomenon is becoming more and more worrying to health authorities, which are multiplying appeals for the development of drug forms for preventing improper use.

### PRIOR ART

To the applicant's knowledge, the only attempts to tackle this problem have consisted in adding, to the medicaments concerned, compounds that are chemically active against misuse.

This solution has certain dangers for uses, including for use under the approved conditions. In addition, combinations of AI and of other active compounds are tricky to control and pose a serious problem to public health.

US-A-2003/0068371 describes an oral pharmaceutical formulation comprising an opiate AI, an antagonist of this AI and a gelling agent (e.g. xanthan gum). The gelling agent is presented as conferring on the formulation a viscosity such that it cannot be administered nasally or parenterally. The presence of this antagonist is a major disadvantage with regard to the medical risks possibly run by users. In addition, this pharmaceutical form can be made into pulverulent form and, consequently, can be the subject of misuse by nasal administration.

### OBJECTIVES OF THE INVENTION

Under these circumstances, one of the essential objectives of the present invention is to overcome the shortcomings of the prior art.

Another essential objective of the invention is to provide novel solid oral medicaments, the misuse of which will be made very difficult or even impossible, in particular for the abovementioned cases (a.)(b.)(c.)(d.), without resorting to substances, other than the AI, that may be pharmaceutically active and therefore dangerous for users.

Another essential objective of the invention is to provide a novel solid oral medicament that makes it possible to prevent the fraudulent misuse of the properties of the AI that it contains, by preventing any conversion of the medicament that means it can be taken orally, nasally and/or by injection (intravenous, subcutaneous, intramuscular, etc.) outside the therapeutic context. In so doing, the risks associated with these derivatives will be prevented or at the very least greatly reduced.

Another essential objective of the invention is to provide a novel solid oral medicament that makes it possible to prevent misuse, while at the same time guaranteeing, for the patient normally followed-up, a quality of treatment, in particular a dose, in accordance with his or her needs.

Another essential objective of the invention is to provide a novel solid oral medicament that makes it possible to prevent misuse without affecting the pharmacological properties of the medicament, and without causing the patient, who uses the medicament normally, to run any additional risks and, finally, without being detrimental to the comfort of the latter during administration.

Another essential objective of the invention is to provide a novel solid oral medicament that makes it possible to prevent misuse, and that is simple to obtain and does not cause its cost price to increase.

### SUCCINCT DESCRIPTION OF THE INVENTION

To attain these objectives, it is to the inventors credit that they have reformulated the general problem of misuse of microparticulate pharmaceutical forms.

If one examines the various illicit methods of administration of an active ingredient, it effectively appears that crushing of the dry microparticulate form is an obligatory step.

In fact, in the case of misuse by parenteral administration, it is necessary to carry out a prior extraction of the AI in a liquid phase, in practice water, and at a concentration sufficiently high to avoid injecting volumes that are too large, for example greater than 1 ml. This extraction step requires prior crushing of the microparticulate dry form in order to allow the active ingredient to be dissolved or suspended.

In the case of misuse by nasal administration, the microparticulate dry pharmaceutical form must be converted beforehand into the form of a pulverulent powder that can be aspirated. Once again, crushing of the microparticulate form is an obligatory step.

In the case of misuse by oral administration of the dry microparticulate form, it is necessary to accelerate the release of the active ingredient by finely crushing the microparticles.

Thus, the crushing of a dry microparticulate form is an obligatory step for misuse of said pharmaceutical form.

It is to the applicant's credit to have reformulated the problem of combating the misuse of dry microparticulate pharmaceutical forms into a problem of prevention of crushing of the microparticles containing the AI.

This new approach has allowed it to discover, surprisingly and unexpectedly, that it is advisable to involve, in the composition of the medicament, the misuse of which it is sought to prevent, a combination of pharmaceutically acceptable excipients for which the physicochemical method of action makes it possible to impede, or even render impossible, any intentional or unintentional act of misuse.

Thus, the invention relates mainly to a solid oral drug form, characterized in that at least part of the AI that it comprises is contained in microparticles, and in that it comprises anticrushing means provided so as to make it difficult, or even impossible, to crush the microparticles of AI, so as to prevent misuse.

The anticrushing means of this dry microparticulate pharmaceutical form are, for example, the excipients comprised therein, which are capable of impeding, or even of rendering impossible (or preventing), the crushing of the microparticles containing the AI.

The drug form according to the invention solves the stated problem and meets the objectives set, in an effective, simple and economical manner, using physicochemical means. The latter are completely harmless for any person using the drug form normally. They are pharmacologically neutral (inert) compounds approved by the pharmacopoeia and by the public health authorities responsible for granting marketing authorizations for medicaments.

### DETAILED DESCRIPTION OF THE INVENTION

The present invention advantageously relates to dry microparticulate pharmaceutical forms, the composition of which makes it possible to prevent, or at the very least make very difficult, the crushing of the microparticles containing the AI.

In a first embodiment of the invention, the anticrushing means comprise at least one caking agent (M).

According to a variant, the drug form according to the invention is free of any combination consisting of at least one caking agent A) and of at least one viscosifying agent B).

In this variant, the caking agent A) is chosen from the class of hydrophobic compounds which act as a dry binder, preferably:
from the group comprising cottonseed oils, soybean oils, palm oils, castor oils and mixtures of all or some of these oils; and/or
from the group of waxes, and even more preferably from the subgroup of waxes comprising hydrogenated cottonseed oils, hydrogenated soybean oils, hydrogenated palm oils, glyceryl behenates, hydrogenated castor oils, tristearins, tripalmitins, trimyristins, yellow waxes, hard fats, anhydrous dairy fats, lanolins, glyceryl palmitostearates, glyceryl stearates, lauric acid macrogolglycerides, cetyl alcohols, polyglyceryl diisostearates, diethylene glycol monostearates, ethylene monostearates, omegas 3 and mixtures of all or some of these waxes; and/or
from the group of fatty bases for suppositories comprising glycerol, triglycerides, theobroma oils, cocoa butters and mixtures of all or some of these products,
   and the viscosifying agent B) is chosen from the following groups of polymers:
polyacrylic acids and derivatives thereof, and/or
polyalkylene glycols (e.g. polyethylene glycol), and/or
polyvinylpyrrolidones, and/or
gelatins, and/or
polysaccharides, preferably from the subgroup comprising: sodium alginate, pectins, guars, xanthans, carrageenans, gellans and cellulose derivatives (e.g. hydroxypropylmethylcellulose, methylcellulose, hydroxyethylcellulose, carboxy-methylcellulose),
   and mixtures thereof.

Preferably, the caking agent (M) is chosen such that, under shear, it is capable of converting the solid drug form into a nonpulverulent paste, making it difficult to crush the microparticles of AI.

Now, it is well known that the extraction of a compound to be concentrated from the viscous paste is extremely difficult.

The caking agent (M) is chosen from the class of hydrophobic compounds which act as a dry binder, preferably:
from the group comprising plant oils: cottonseed oils, soybean oils, palm oils, castor oils and mixtures of all or some of these oils; and/or mineral oils and/or
from the group of waxes, and even more preferably from the subgroup of waxes comprising hydrogenated cottonseed oil waxes, hydrogenated soybean oils, hydrogenated palm oils, glyceryl behenates, hydrogenated castor oils, tristearins, tripalmitins, trimyristins, yellow waxes, hard fats, anhydrous dairy fats, lanolins, glyceryl palmitostearates, glyceryl stearates, lauric acid macrogolglycerides, cetyl alcohols, polyglyceryl diisostearates, diethylene glycol monostearates, ethylene monostearates, omegas 3 and mixtures of all or some of these waxes; and/or
from the group of fatty bases for suppositories comprising glycerol, triglycerides, theobroma oils, cocoa butters and mixtures of all or some of these oils.

In a second embodiment of the invention, the microparticulate drug form is characterized in that its anticrushing means comprise insoluble inert microbeads which have an average diameter of greater than or equal to 1.25 times, preferably 1.5 times, and even more preferably twice, the average diameter of the microparticles of AI.

These microbeads which are insoluble in an aqueous or aqueous-alcoholic medium, cannot be compressed. Due to the fact that the neutral beads are larger than the microparticles containing the AI, the crushing stresses are mainly borne by the neutral beads, thus protecting the microparticles containing the AI. They therefore make attempts at mechanical crushing ineffective.

Preferably, the insoluble neutral microbeads are chosen from the group of following substances: celluloses and insoluble derivatives thereof, polymethacrylic resins and derivatives thereof, silicas, talc, semolina, bentonite and mixtures thereof.

In a third embodiment of the invention, the microparticulate drug form is characterized in that its anticrushing means comprise at least one lubricant (L).

The role of the lubricant(s) is to greatly limit, or even eliminate, the abrasion of the microparticles containing the AI when they are mechanically crushed. The lubricant or slip agent makes it difficult to crush the multiparticulate drug form by facilitating its flow, thus reducing the shear stress applied to the product.

The advantage of the lubricant or slip agent is to generate wall slip; the product does not therefore adhere to the wall of the mill, thereby preventing transmission of the shear stress to the active ingredient present in the microparticles.

In the presence of lubricant or slip agent, the powder composed of microparticles of active ingredient is not cohesive and has a very good flow.

The lubricant is either simply mixed with the solid microparticulate pharmaceutical form, or else is directly deposited at the surface of the microparticles containing the AI by any means known to those skilled in the art, for example by spray-coating.

By way of nonlimiting examples of lubricants or slip agents, mention may be made of:
calcium stearate;
glyceryl palmitostearate;
hydrogenated plant oils;
magnesium oxide;
poloxamers;
polyethylene glycols;
polyvinyl alcohol;
sodium benzoate;
anionic, cationic or nonionic surfactants;
stearic acid;
corn starch;
talc;
colloidal silica;
zinc stearate, magnesium stearate,
and mixtures thereof.
   The fraction by mass of lubricant in the solid pharmaceutical form according to the invention is, for example, between 1% and 40%.

The microparticles according to the invention and which contain the AI have an average diameter of less than or equal to 1000 microns, preferably between 50 and 800 microns, and more preferably between 100 and 600 microns.

The microparticles according to the invention and which contain the AI may be microcapsules for modified release of AI, i.e. microparticles coated with a polymer film deposited according to techniques known to those skilled in the art. On this subject, the article “formes pharmaceutiques nouvelles” [“new pharmaceutical forms”] by Buri, Puisieux, Doelker and Benoit, Lavoisier 1985, p 175-227, will for example be consulted.

Advantageously, the drug form according to the invention cannot be converted into a dry form that can be administered by nasal aspiration.

It should also be noted that this drug form according to the invention cannot be converted into an injectable form.

According to a variant, the drug form according to the invention comprises immediate-release AI and/or modified-release AI.

Preferably, at least some of the microparticles of AI of the drug form according to the invention are microparticles, preferably microcapsules, for modified release of AI.

Advantageously, the microparticles of AI or the microcapsules of AI have an average diameter of less than or equal to 1000 μm, preferably between 50 and 800 microns, and even more preferably between 100 and 600 microns.

According to another notable characteristic of the drug form according to the invention, extraction of the AI by chewing and/or crushing is ineffective.

The AI used belongs, for example, to at least one of the following families of active substances: amphetamines, analgesics, appetite suppressants, antidepressants, antiepileptics, antimigraine agents, antiparkinsonian agents, antitussives, anxiolytics, barbiturates, benzodiazepines, hypnotics, laxatives, neuroleptics, opiates, psychostimulants, psychotropic agents, sedatives and stimulants.

Even more specifically, the AI used is chosen from the following compounds:
methylphenidate, Fentanyl, Alfentanyl, Pentazocine, Pethidine, Phenoperidine, Remifentanil, Sufentanil, Acetorphine, Acetylalphamethylfentanyl, Acetylmethadol, Alfentanil, Allylprodine, Alphacetylmethadol, Alphameprodine, Alphamethadol, Alphamethylfentanyl, Alpha-methylthiofentanyl, Alphaprodine, Anileridine, atropine, Benzethidine, Benzylmorphine, Beta-hydroxyfentanyl, Beta-hydroxymethyl-3-fentanyl, Betacetylmethadol, Betameprodine, Betamethadol, Betaprodine, Bezitramide, buprenorphine, Dioxaphetyl butyrate, Cannabis, Ketobemidone, Clonitazene, codeine, Coca, Cocaine, Codoxime, Concentrate of poppy straw, Desomorphine, Dextromoramide, Diampromide, Diethylthiambutene, Difenoxine, Dihydroetorphine, Dihydromorphine, Dimenoxadol, Dimepheptanol, Dimethylthiambutene, Diphenoxylate, Dipipanone, Drotebanol, Ecgonine, ephedrine, Ethylmethylthiambutene, Etonitazene, Etorphine, Etoxeridine, Fentanyl, Furethidine, Heroin, Hydrocodone, Hydromorphinol, Hydromorphone, Hydroxypethidine, Isomethadone, Levomethorphane, Levomoramide, Levophenacylmorphane, Levorphanol, meperidine, Metazocine, Methadone, Methyldesorphine, Methyldihydromorphine, Methyl-3-thiofentanyl, Methyl-3-fentanyl, Metopon, Moramide, Morpheridine, morphine, MPPP, Myrophine, Nicomorphine, Noracymethadol, Norlevorphanol, Normethadone, Normorphine, Norpipanone, Opium, Oxycodone, Oxymorphone, Para-fluorofentanyl, PEPAP, Pethidine, Phenampromide, Phenazocine, Phenomorphane, Phenoperidine, Piminodine, Piritramide, Proheptazine, propanolol, Properidine, Racemethorphane, Racemoramide, Racemorphane, Remifentanil, Sufentanil, Thebacone, Thebaine, Thiofentanyl, Tilidine, Trimeperidine, Acetyldihydrocodeine, Codeine, Dextropropoxyphene, Dihydrocodeine, Ethylmorphine, Nicocodine, Nicodicodine, Norcodeine, Pholcodine, Propiram,
   and mixtures thereof.

The above embodiments can be taken in isolation or in combination with one another.

### EXAMPLE 1

### Preparation of Microparticles of Acyclovir

### Model Active Ingredient

Step 1: Granules

45 g of acyclovir, 25 g of PEG-40-hydrogenated castor oil and 30 g of povidone are solubilized beforehand in a water/acetone/isopropanol mixture (5/57/38 m/m). This solution is then sprayed onto 800 g of cellulose spheres (of diameter between 100 and 200 μm) in a Glatt GPC-G1 fluidized airbed apparatus.

Step 2: Coating

50 g of previously obtained granules are coated with 6.5 g of ethylcellulose, 0.5 g of castor oil, 0.5 g of PEG 40-hydrogenated castor oil (BASF) and 2.5 g of povidone dissolved in an acetone/isopropanol mixture (60/40 m/m), in a miniGlatt fluidized airbed apparatus.

The mean diameter of the microparticles obtained is 180 μm. These microparticles are virtually spherical.

### EXAMPLE 2

35 g of PEG 6000 and 5 g of magnesium stearate are dispersed in 160 g of isopropanol. This dispersion is then sprayed onto 40 g of microparticles obtained at the end of the second step of example 1.

When these objects are subjected to shear, the layer containing the PEG 6000 and the magnesium stearate protects the particles of active ingredient by reducing the effects of the shear.

What is claimed is:

1. A solid oral drug form, characterized in that at least part of the AI that it comprises is contained in microparticles, and in that it comprises anticrushing means provided so as to make it difficult, or even impossible, to crush the microparticles of AI, so as to prevent misuse.

2. The drug form as claimed in claim 1, characterized in that the anticrushing means are excipients which go to make up the composition of the drug form and which are capable of impeding, or even preventing, crushing of the microparticles of AI.

3. The drug form as claimed in claim 1 or 2, characterized in that it consists of a dry form.

4. The drug form as claimed in any one of the preceding claims, characterized in that the anti-crushing means comprise at least one caking agent (M).

5. The drug form as claimed in claim 4, characterized in that it is free of any combination consisting of at least one caking agent A) and of at least one viscosifying agent B).

6. The drug form as claimed in claim 5, characterized in that the caking agent A) is chosen from the class of hydrophobic compounds which act as a dry binder, preferably:
from the group comprising cottonseed oils, soybean oils, palm oils, castor oils and mixtures of all or some of these oils; and/or
from the group of waxes, and even more preferably from the subgroup of waxes comprising hydrogenated cottonseed oils, hydrogenated soybean oils, hydrogenated palm oils, glyceryl behenates, hydrogenated castor oils, tristearins, tripalmitins, trimyristins, yellow waxes, hard fats, anhydrous dairy fats, lanolins, glyceryl palmitostearates, glyceryl stearates, lauric acid macrogolglycerides, cetyl alcohols, polyglyceryl diisostearates, diethylene glycol monostearates, ethylene monostearates, omegas 3 and mixtures of all or some of these waxes; and/or
from the group of fatty bases for suppositories comprising glycerol, triglycerides, theobroma oils, cocoa butters and mixtures of all or some of these products,
and in that the viscosifying agent B) is chosen from the following groups of polymers:
polyacrylic acids and derivatives thereof, and/or polyalkylene glycols (e.g. polyethylene glycol), and/or
polyvinylpyrrolidones, and/or
gelatins, and/or
polysaccharides, preferably from the subgroup comprising: sodium alginate, pectins, guars, xanthans, carrageenans, gellans and cellulose derivatives (e.g. hydroxypropylmethylcellulose, methylcellulose, hydroxyethylcellulose, carboxy-methylcellulose),
and mixtures thereof.

7. The drug form as claimed in claim 4, characterized in that the caking agent (M) is chosen such that, under shear, it is capable of converting the solid drug form into a nonpulverulent paste making it difficult to crush the microparticles of AI.

8. The drug form as claimed in claim 4 or 7, characterized in that the caking agent (M) is chosen from the class of hydrophobic compounds which act as a dry binder, preferably:
from the group comprising cottonseed oils, soybean oils, palm oils, castor oils and mixtures of all or some of these oils; and/or
from the group of waxes, and even more preferably from the subgroup of waxes comprising hydrogenated cottonseed oils, hydrogenated soybean oils, hydrogenated palm oils, glyceryl behenates, hydrogenated castor oils, tristearins, tripalmitins, trimyristins, yellow waxes, hard fats, anhydrous dairy fats, lanolins, glyceryl palmitostearates, glyceryl stearates, lauric acid macrogolglycerides, cetyl alcohols, polyglyceryl diisostearates, diethylene glycol monostearates, ethylene monostearates, omegas 3 and mixtures of all or some of these waxes; and/or
from the group of fatty bases for suppositories comprising glycerol, triglycerides, theobroma oils, cocoa butters and mixtures of all or some of these products.

9. The drug form as claimed in one of the preceding claims, characterized in that the anticrushing means comprise insoluble inert microbeads which have an average diameter of greater than or equal to 1.25 times, preferably 1.5 times, and even more preferably twice, the average diameter of the microparticles of AI.

10. The drug form as claimed in claim 9, characterized in that the insoluble neutral microbeads are chosen from the group of following substances: celluloses and insoluble derivatives thereof, polymethacrylic resins and derivatives thereof, silicas, talc, semolina, bentonite and mixtures thereof.

11. The drug form as claimed in any one of the preceding claims, characterized in that the anti-crushing means comprise at least one lubricant (L).

12. The drug form as claimed in claim 11, characterized in that the lubricant (L) is chosen from those capable of eliminating the abrasion of the microparticles containing the AI when they are mechanically crushed.

13. The drug form as claimed in claim 12, characterized in that the lubricant (L) is chosen from the group comprising:
calcium stearate;
glyceryl palmitostearate;
hydrogenated plant oils;
magnesium oxide;
poloxamers;
polyethylene glycols;
polyvinyl alcohol;
sodium benzoate;
anionic, cationic or nonionic surfactants;
stearic acid;
corn starch;
talc;
colloidal silica;
zinc stearate, magnesium stearate,
and mixtures thereof.

14. The drug form as claimed in any one of the preceding claims, characterized in that it cannot be converted into a dry form that can be administered by nasal aspiration.

15. The drug form as claimed in any one of the preceding claims, characterized in that it cannot be converted into an injectable form.

16. The drug form as claimed in any one of the preceding claims, characterized in that it comprises immediate-release AI and/or modified-release AI.

17. The drug form as claimed in any one of the preceding claims, characterized in that at least some of the microparticles of AI are microparticles, preferably microcapsules, for modified release of AI.

18. The drug form as claimed in any one of the preceding claims, characterized in that the microparticles of AI or the microcapsules of AI have an average diameter of less than or equal to 1000 μm, preferably between 50 and 800 microns, and even more preferably between 100 and 600 microns.

19. The drug form as claimed in any one of the preceding claims, characterized in that extraction of the AI by chewing and/or crushing is ineffective.

20. The drug form as claimed in one of the preceding claims, characterized in that the AI used belongs to at least one of the following families of active substances: amphetamines, analgesics, appetite suppressants, antidepressants, antiepileptics, anti-migraine agents, antiparkinsonian agents, antitussives, anxiolytics, barbiturates, benzodiazepines, hypnotics, laxatives, neuroleptics, opiates, psychostimulants, psychotropic agents, sedatives and stimulants.

21. The drug form as claimed in one of the preceding claims, characterized in that the AI used is chosen from the following compounds:
methylphenidate, Fentanyl, Alfentanyl, Pentazocine, Pethidine, Phenoperidine, Remifentanil, Sufentanil, Acetorphine, Acetylalphamethylfentanyl, Acetylmethadol, Alfentanil, Allylprodine, Alphacetylmethadol, Alphameprodine, Alphamethadol, Alphamethylfentanyl, Alpha-methylthiofentanyl, Alphaprodine, Anileridine, atropine, Benzethidine, Benzylmorphine, Beta-hydroxyfentanyl, Beta-hydroxymethyl-3-fentanyl, Betacetylmethadol, Betameprodine, Betamethadol, Betaprodine, Bezitramide, buprenorphine, Dioxaphetyl butyrate, Cannabis, Ketobemidone, Clonitazene, codeine, Coca, Cocaine, Codoxime, Concentrate of poppy straw, Desomorphine, Dextromoramide, Diampromide, Diethylthiambutene, Difenoxine, Dihydroetorphine, Dihydromorphine, Dimenoxadol, Dimepheptanol, Dimethylthiambutene, Diphenoxylate, Dipipanone, Drotebanol, Ecgonine, ephedrine, Ethylmethylthiambutene, Etonitazene, Etorphine, Etoxeridine, Fentanyl, Furethidine, Heroin, Hydrocodone, Hydromorphinol, Hydromorphone, Hydroxypethidine, Isomethadone, Levomethorphane, Levomoramide, Levophenacylmorphane, Levorphanol, meperidine, Metazocine, Methadone, Methyldesorphine, Methyldihydromorphine, Methyl-3-thiofentanyl, Methyl-3-fentanyl, Metopon, Moramide, Morpheridine, morphine, MPPP, Myrophine, Nicomorphine, Noracymethadol, Norlevorphanol, Normethadone, Normorphine, Norpipanone, Opium, Oxycodone, Oxymorphone, Para-fluorofentanyl, PEPAP, Pethidine, Phenampromide, Phenazocine, Phenomorphane, Phenoperidine, Piminodine, Piritramide, Proheptazine, propanolol, Properidine, Racemethorphane, Racemoramide, Racemorphane, Remifentanil, Sufentanil, Thebacone, Thebaine, Thiofentanyl, Tilidine, Trimeperidine, Acetyldihydrocodeine, Codeine, Dextropropoxyphene, Dihydrocodeine, Ethylmorphine, Nicocodine, Nicodicodine, Norcodeine, Pholcodine, Propiram, and mixtures thereof.
